(19) United States
(12) Patent Application Publication
(10) Pub. No.: US 2005/0003535 A1
(43) Pub. Date: Jan. 6, 2005

(54) Bioreactor for cell self-assembly in form of an organ copy; procedures for the production and the application of cell culture, differentiation, maintenance, proliferation and/or use of cells

(76) Inventor: Joerg C. Gerlach, Pittsburgh, PA (US)

Correspondence Address: Joerg C. Gerlach MD, PhD, 3613 Butler St., Pittsburgh, PA, 15201, US

(21) Appl. No.: 10/866,221

(22) Filed: Jun. 12, 2004

(30) Foreign Application Priority Data
Jun. 13, 2003 (DE) 103 26 746.8

(51) Int. Cl. C12M 3/00

(52) U.S. Cl. 435/396; 435/299.1; 435/1.1

(57) ABSTRACT

The invention concerns a bioreactor in form of an organ copy representing the typical structures of animal or human organs; a procedure to manufacture as well as use the bioreactor for the cultivation, differentiation, maintenance, proliferation and/or use of organ cells or stem cells. The characteristic of this invention is that the specific hollow pathway structures supplying the cells in the open pore body are the exact same configuration as they occur in a natural organ. A further characteristic is the cell culture within open-porous structures, being perfused between the hollow pathway structures, branching out from the center to the periphery and branching in from the periphery to the center. With this bioreactor a device is described that facilitates the reorganization and use of microorganisms or cells in a manner typical to that of the natural organ.

The invention concerns a bioreactor in form of an organ copy representing the typical structures of animal or human organs; a procedure to manufacture as well as use the bioreactor for the cultivation, differentiation, maintenance, proliferation and/or use of organ cells or stem cells. The characteristic of this invention is that the specific hollow pathway structures supplying the cells in the open pore body are the exact same configuration as they occur in a natural organ. A further characteristic is the cell culture within open-porous structures, being perfused between the hollow pathway structures, branching out from the center to the periphery and branching in from the periphery to the center.

The use of bioreactors to culture cells, e.g. Petri dishes, cell perfusion systems or reaction vessels are well-known in the industry, especially in the areas of: clinical therapy with cells, the production of biological cells or cell products, as an alternative method to animal experiments or for the usage of cell performance. Today, bioreactor technology, cultivation and multiplication of cells is well known.

Such a bioreactor is described in WO 0075275 (Mac Donald, USA/EP 1185612 (Mac Donald, USA). These systems, so far, have not yielded satisfactory results in regards to the performance of primary cells and stem cells in comparison to the natural organ with its vascular system. At present, it is especially not possible to isolate various cells including stem cells from an organ and reorganize them “in vitro” to a larger cell mass under organ conditions typical to their natural state.

Consequently, it is the purpose of the above mentioned invention to describe a bioreactor that, based on its structure and capacity, has the ability to simulate natural organs and its stem cell components as close as possible to nature's intent. In addition, the purpose of this invention is to give a process to manufacture and use such bioreactor.

In regards to the bioreactor this task will be achieved through the identifying features of patent claim 1, and for the manufacturing processes through the identifying features of patent claim 12. The underlying claims point out further advantages, and developments. Claims 27 through 41 state additional uses of the bioreactor.

The core point of the invention, as per patent claim 1, is that the natural hollow structures of an organ, often being the vascular systems but also the stem cell/progenitor cell compartments, are replicated with the supply and waste disposal structures identical to the natural organ. Examples of the natural hollow structures an organ, at the example of the liver include: supplying arteries, discharging veins, as well as typical organ specific vessels like the portal veins of the liver, vessels of the biliary system, and the canals of Hering with liver stem cells. These structures will be achieved by producing a bioreactor in form of an organ copy.

The hollow structures of the bioreactor permit the supply of a large, highly dense cell mass in the organs. The exchange of fluids via blood plasma or media occurs in a decentralized manner, e.g. between arteries and veins avoiding large substance gradients.

It is essential, that the porous part of the bioreactor contains open pores that have the ability to communicate with each other. This basic structure of the bioreactor correspond to afore mentioned tissue of the natural organs between arteries and veins. The porous part of the bioreactor is made of immunological inactive material. These open pores are of larger size than the cells of the respective organs. Therefore, the preferred diameter of the pores lies in the range of 10-1000 micrometer. The pores are connected through openings in the pore walls. These openings are preferably in the shape of canals and about 5-500 micrometer in size. This concept assures that, via the pore openings, the pores are in constant exchange with each other and the organ copies of the hollow structures. The above-described structure of the porous body can also be described as an open-porous foam/sponge-like structure.

The invention of this bioreactor describes a device that allows organ-typical reorganization and culture of biological cells. The characteristic of this invention is that the specific hollow pathway structures supplying the cells in the open pore body are the exact same configuration as they occur in a natural organ. A further characteristic is the cell culture within open-porous structures, being perfused between the hollow pathway structures, branching out from the center to the peripheri and branching in from the peripheri to the center.

It is important that the bioreactor consists of a perfuseable, open-porous, foam/sponge-like structure where the cells are imbedded around the hollow structures and the pores of the construction are in communication with each other. Through theses pores the following functions are made possible: flushing of cells into the pores, medium perfusion of cells, cell migration, cell re-assembly, cell proliferation, cell differentiation, and cell metabolism. As a result, the above described bioreactor, realizes a development that is in regards to its metabolic structure far superior to the characteristics and performance of currently used bioreactors.

All materials, producing open-pore structures, currently known to man, and used in technology, are suitable materials for the open porous foam/sponge-like structure for the purpose of this invention. For example, ceramics like Hydroxyapatite are already well known and researched in medicine and are therefore well suited for this application. Hydroxyapatite is available as a powder. It can be, if necessary, used as a suspension with the addition of foam/bubble and pore producing substances and other additives, frothed to the desired foam/sponge structure and then sintered. Additives like proteins that are used as organic foaming agents are burnt-at in high temperature sintering processes. Preferred additives for this process are foam producing materials, such as baking soda, which during the process of increasing temperature, become gaseous and brake up the dividing walls of the foam blisters.

The bioreactor is preferably embedded in a germ- and watertight environment.

Suitable environments include: foils or other appropriately sized containers. In this case the container is fitted with connections that are in contact with at least one hollow structure of the organ to ensure supply and waste removal in the bioreactor. In regards to the construction of the connections, it is possible to connect several inputs and/or output of the organ to a single input and/or output of the container. Such solutions for bioreactors are already well known from, e.g. WO 0075275 (Mac Donald), USA/EP 1185612 (Mac Donald, USA).

In another design the container shows additional connections. One connection serves to fill microorganism cells into the bioreactor. Other connections are used to measure pressure, ph-levels, temperature, insert probes for microscopy, or take measurements inside the module via fluorescent light processes. Further parts may lead to the application of movements or pressure to facilitate cell harvest.

An additional variation of the container allows for the sterile opening of the case to remove individual parts of the porous structure that are populated by cells, e.g. individual previously prepared discs, for the purpose of microscopy and/or for clinical implantation in regenerative medicine.

Furthermore, the housing may incorporate microscopy glass cover slips for online (video) microscopy through the housing.

It is also advantageous, that the case and the connections of the bioreactor can be manufactured from absorbable, biodegradable material that allows the entire bioreactor or parts of it to be used as an implant.

Manufacturing of the bioreactor is preferred for copies of the following organs: bone marrow, lymph nodes, thymus gland, spleen, kidney, liver, pancreas, Islets of Langerhans, mucus membranes, thyroid, parathyroid glands, adrenal glands, bones, testis, uterus, placenta, ovaries, blood vessels, heart, lungs, muscle, heart muscle, intestine, bladder, or other mammalian organs.

The inventors were able to demonstrate that a bioreactor in form of an organ copy possesses excellent structures. The cells that are flushed into the bioreactor are immobilized inside the open pores to reorganize themselves according to nature's intent. The cells can be well supplied in a densely populated environment, as well as reorganized into tissue structures via the use of co-cultures of parenchymal cells and organ typical non-parenchymal cells.

In early experiments on the liver (see figure) the inventors were able to demonstrate that the above described organ copy technology resulted in excellent results. Therefore, a bioreactor in form of a copy of the liver presents an excellent execution of the invention. Naturally, this invention is not limited to copying the liver organ, but is generally transferable to other organs, e.g. bone marrow.

The invention is also relevant to the manufacturing process of the above described organ copy bioreactor. This process can also be described as an organ structure positive/negative casting process.

The manufacturing process of the casting is based on the procedures a) through e) described in patent claim 12. The procedures a) and c) introduce substances while the procedures b) and d) remove substances. The biological substances and the organ cells are completely dissolved within the negative/positive casting process, while the supplying hollow structures of the organ are being copied. The biological walls of the natural hollow structures are replaced by immunological inactive, open pore foam structures. The cells can reorganize themselves in the open pores between the hollow structures. During this process the immunological characteristics of the original organ completely disappear. That includes the original metabolic characteristics of the original organ (e.g. porcine liver metabolism) that in turn can be replaced by the succeeding cells (e.g. human liver cells for the human liver metabolism).

The first step a) fills up the organ's hollow structures around which the organ cells are arranged. This preferably occurs separately across all supplying and removing structures. Step a) creates a three-dimensional negative cast of the vessel, e.g. blood vessel, and reproduces the architecture of all supply and removal structures. It is important that the negative cast consist of a material that survives the succeeding digestion of the original biological organ material, and which remains mechanically stabile enough to permit a new positive cast to be made with an open pore foam like-structure. In addition, the negative cast material has to be such that, after filling up the hollow structures with the open pore foam material, it can be completely dissolved or evaporated without destroying the open pore foam structure of the cast. Liquid synthetic materials or polymers that can easily be infiltrated into the hollow structures and then solidified are preferred for this process. Two component polymers, well known in the field of anatomy, are suitable. The chosen material for the negative copies will polymerize and crosslink inside the hollow structures. In general, any materials with a maximum decomposition/evaporization temperature of 600 degrees Celsius would be suitable as well as liquid synthetic materials in the form of single-component or two-component materials or polymers.

In step b) the digestion of the cells and the connective tissue structures of the biological organ take place. Digestive enzymes like collagenase/Trypsin can be used. Equally useful for the dissolution of tissue material are acid or base rinses or alternating acid/base rinses if the substance of the negative cast is suitable for such treatment. Step A and B can be performed with any material/process that allows positive/negative casting. This may include, for example, the use of crystal foaming agents in step A and high temperature burning in step B.

In step c) a positive cast with an open-pore, sponge-/foam-like material is made that takes the place within the original organ, in the area between the original vessel structures. Characteristic for the choice of the open-pore, sponge-like forming material is that the sponge-like porous areas communicate with each other and the positive/negative cast cell supplying structures/vessels, and that they can accommodate the cells. These pores also allow free passage of 1. cells (during infiltration and cell migration), and 2. free circulation of culture medium/metabolites of the cells.

Ceramics, such as hydroxyapatite, with open-pore foaming additives is the preferred substance for the positive cast. Hydroxyapatite belongs to the group of calcium phosphate substances, which includes ceramic materials with different fractions of calcium and phosphor. Hydroxyapatite is a chemical compound that exists in nature but can also be synthetically manufactured. The use of hydroxyapatite is already a state of the art in the medical community. The motivation for the clinical use of hydoxyapatite is the application of a substance with similar chemical composition to the mineral part of the bone and around the bone marrow stem cells. Hydroxyapatite makes up 60-70% as a natural component in the mineral portion of the bone.

Hydroxyapatite powder is generated through the process of precipitation from an aqueous solution with the addition of, for instance, ammonium phosphate to a calcium nitrate solution under alkaline pH conditions. The powder particles can be bonded through a sintering process at 1000-1600 degrees Celsius. Additives lead to the forming of open pores in the cast prior to sintering.

Wintermantel describes an example of the manufacture of porous, solid structures made of hydroxyapatite, e.g. open-pore, foam like structures in which hydroxyapatite powder is mixed with organic additives, such as protein albumin and baking soda, that will later burn away at high temperatures. Thereby it is preferred to use foam producing substances that become gaseous under increasing temperature and during this process brake open the dividing walls between the foam blisters (Wintermantel E, Suk-woo Ha: Bio-compatible substances and building elements: implants for medicine and environment. Berlin/Springer 1998:256-257; ISBN 3-540-64656-6).

In step d) the substance of the negative copy is removed which creates hollow structures that take the place of the vessels and the other cast structures of the biological organs. During the removal of the substances, by decomposition or evaporation, the negative cast of the organ has to be removed without severely altering the structure of the positive cast.

The bioreactor, developed according to steps a) through d) will then be placed into a sterile and watertight container. Suitable containers would include foils and/or similar containers. The chosen container is equipped with connections, of which at least one is in connection with the hollow structure of the organ copy, to ensure the supply of media and removal of media, from the growing tissue, as well as the injection of cells.

For the dissolution of organ tissue (dissolution step b), and the dissolution of the negative cast materials (dissolution step d), opposing properties of the open-pore foam structure material of the positive-and negative cast is crucial. In addition to the described materials and techniques, any material/technique can be applied. After forming the positive cast between the negative material, the negative material has to be completely removable without changing or altering the structure of the positive cast.

Examples for various technical solutions to this problem are described in the example of the use of open-pore, ceramic-foam materials as positive cast material.

Since hydroxyapatite is heat resistant, a polymer can be used as negative cast material, which completely burns/evaporates at temperatures above 600 degrees Celsius. Hydroxyapatite is not water soluble. Therefore, a crystallizing substance can be used for the positive cast, which will dissolve during the process of submerging the hydroxyapatite into liquid solution. Similar results can be achieved by using natural or artificial wax, that are injected in liquid form above melting temperature to cool down/solidify inside the organ's vessel structure and as a result survive the organic material's decay process.

Comparable results can be achieved using materials of opposing properties, for instance materials exhibiting different characteristics at ph-changes, or materials exhibiting different characteristics after adding solvents, etc. A further example would be to perfuse the organ with a substance, which covers tissue structure but does not enter into the cells. After solidification of the substance and subsequent destruction of the cells, e.g. through acid/base treatment, ceramic suspension may be perfused to coat afore mentioned structures, and subsequently allow for the application of the procedures described above. Such techniques, to be applied in combination with the invention, are known from other fields of ceramic powder applications. This includes, e.g. converting a native biopolymeric material into ceramic products by pyrolytic decomposition, resulting into a template (carbon replica), which subsequently can react to or be infiltrated to yield oxide reaction products. Furthermore, infiltration of chemically preprocessed natural materials with gaseous or liquid precursors and subsequent oxidation is known.

The process of manufacturing a perfuseable body in the sense of a closed culture system allow for the attachment of sterile containers like exterior cases, connections for the supply and waste removal of culture media, blood, plasma, or cell products and other devices. Additional accesses ways could be included, for instance, to measure pressure, pH, and temperature, or to insert optical probes for the purpose of microscopy, or to take measurements inside the bioreactor via fluorescent light technique.

A design feature of this invention is the option to manufacture these connections, the materials of the container, and the open-pore foam/sponge- like bioreactor material from absorbable/biodegradable materials in such a way that clinical implantation of the entire structure is possible. This could lead to a permanent biological organ replacement with perfuseable cell structures in a patient. Latter is possible when immunological compatible cells are cultivated (e.g. autologous cells, stem cells or trans-genetic cells). Beneficial for the implantation of such structures is the fact that with this invention all biological components of the originally cast organ disappear. This allows the use of cast organs originating from animals in step a).

Further steps could be used to culture cells and to self-assemble cells in the open-pore foam like structures of the body. Specifically, a cleaning process with media and a sterilizing process is possible. A coating with biomatrix, e.g. collagen is considered a state of the art procedure. By using biomatrix producing cells in co-culture in the body, a coating with foreign biomatrix can be avoided.

Because of the high cell density in the body, sufficient oxygen supply is desired which can be achieved through high circulation rates of oxygenized media as well as also via oxygen carriers circulating with the media (e.g. synthetic hemoglobin or erythrocytes).

Other adaptations could be made to facilitate state of the art applications of culture systems. The cells are flushed in through the hollow structures that correspond to the vessels of the original organ. This typically occurs with all organ typical cells for a co-culture of all cells of the organ. The flushing of the cells occurs with culture media.

After the organ's parenchyma and non-parenchyma cells were flushed in, an organ typical culture, e.g. by self-reassembling of non-parenchymal cells and parenchymal cells can occur inside the bioreactor. This is well-known for hepatocytes and sinusoidal endothelial cells, and stem cells of the liver.

Some organs produce cells that are later on flushed out via the blood stream, e.g. bone marrow stem cells. Analogous, a flushing of proliferating cells from the bioreactor, for example immune cells, blood cells, and/or stem cells, could facilitate cell harvest.

Cell harvesting can be achieved through flushing with culture media, if need be after enzymatic biomatrix digestion with collagenas/Trypsin, and/or in combination with the application of movements and/or forces.

If a custom made device is to be implanted into a patient's body and the surgical connections of the hollow structures be connected to the patient's blood vessels, it is possible to the seed vascular endothelial cells (e.g. the patient's own vascular cells) into the hollow structures prior to implantation. This improves the compatibility with the blood after implantation and reduces the formation of blood clots.

The use of the patient's own endothelial cells would reduce immune reactions.

With this described method, adult liver stem cells, human bi-potential progenitor cells, have been successfully cultivated into liver structures.

The description of the invention continues to include the use of the above-described bioreactor.

In general, the invented bioreactor is suitable for the culture, preservation, maintenance, proliferation, and/or use of individual cells and various kinds of cells (co-culture) of an organ, cell lines, gene technologically modified cells, or immortalized cells. The bioreactor can be used in industry by using cells to produce diagnostic/therapeutic substances, but also for the production of cells for industrial or therapeutic use as well as for therapeutic transplantation. The bioreactor can also be used to utilize cell performances for a patient in the sense of an extracorporeal temporary hybrid organ support system. Additionally, the bioreactor can be used to culture implantable organ transplants. The bioreactor is also suitable as a laboratory device to replace or supplement animal testing in research and in the pharmaceutical industry. Another possibility is to utilize the bioreactor for the creation of a cell system to propagate viruses like HIV and hepatitis B/C viruses. The bioreactor can also be utilized for the production of vaccines. Finally, the device is especially suitable for the reorganization and preservation of stem cells, their growth, and differentiation toward organ tissue.

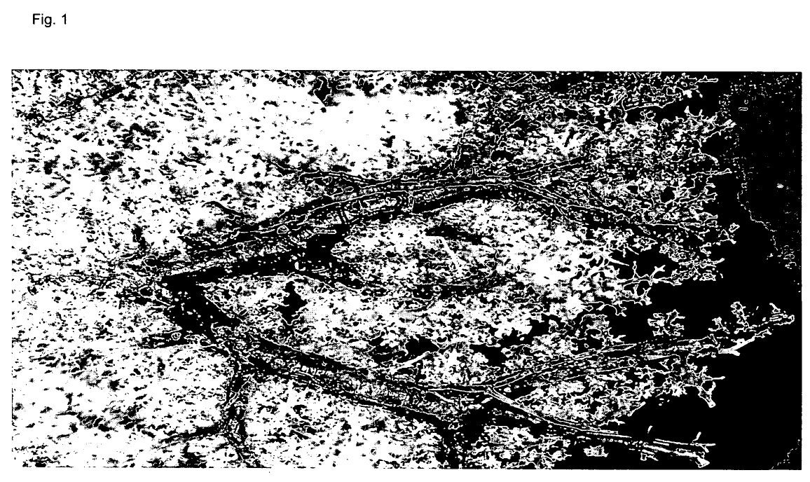

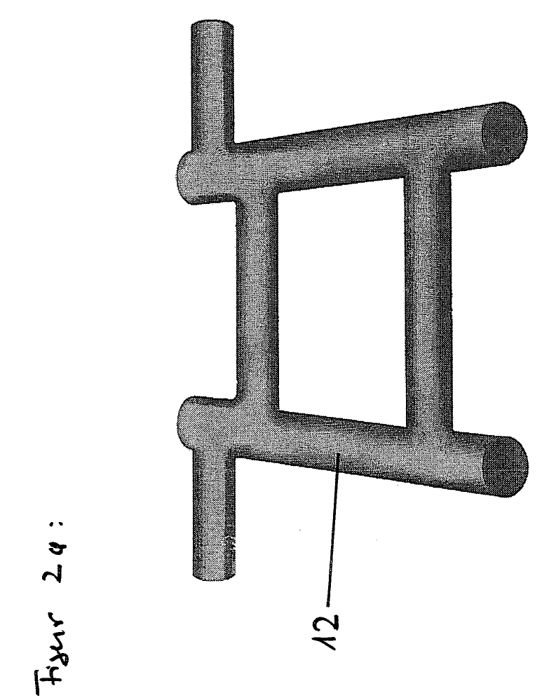

The invention will be further described in FIG. 1 through 4.

FIG. 1 illustrates the negative copy of a liver's vessel structure. In order to manufacture the structure shown in FIG. 1 all vessel structures of an organ, in this case the liver, are canulated and injected with a liquid two-component synthetic polymer. The injected substance will polymerize inside the organ's vessel structures and solidify. To create the negative copy of the organ's vessel structure, as shown in FIG. 1, the organic materials were removed through acid treatment. FIG. 1 demonstrates that such a procedure not only replicates the arteries and veins, but also the most delicate capillary structures of the organ.

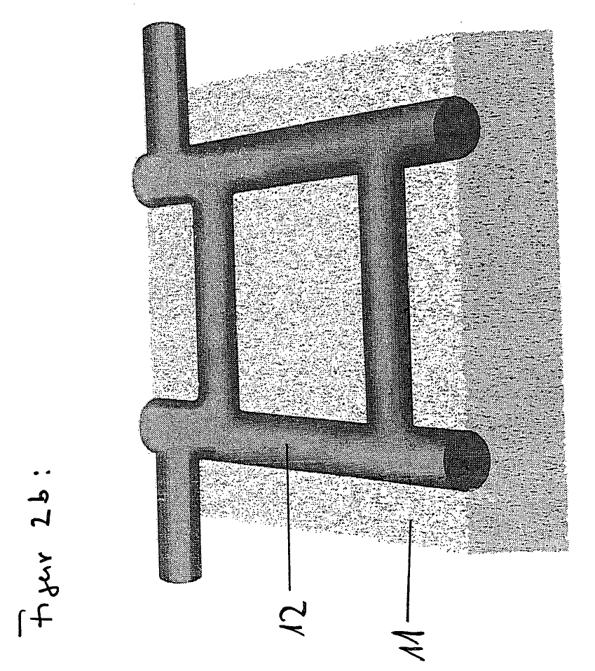

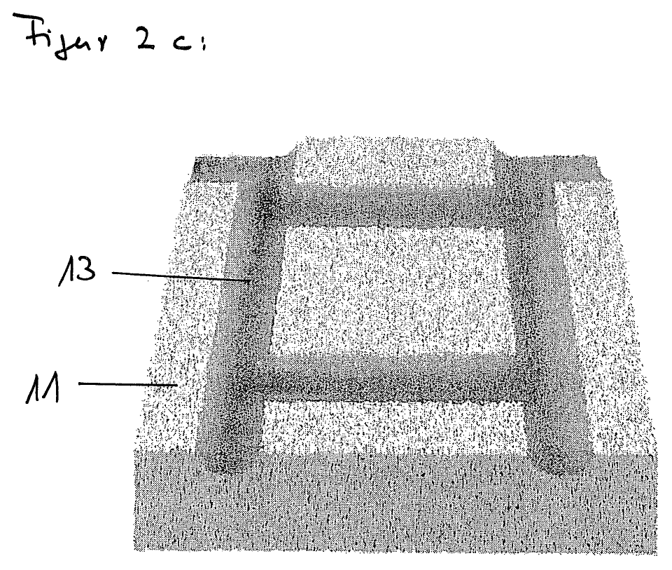

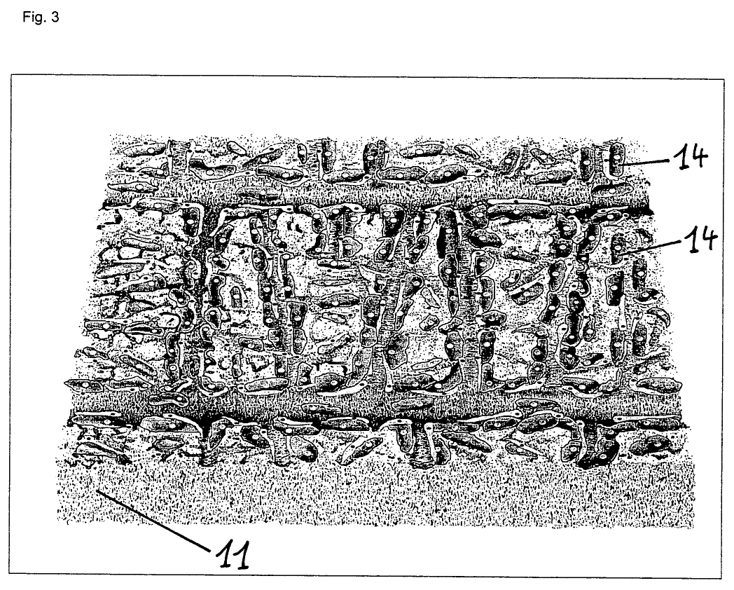

FIG. 2a-c, illustrates the steps of the manufacturing process of the organ copy. FIG. 2a shows, with the aid of an example, processing after the removal of all organic materials. Therefore FIG. 2a shows the process described in FIG. 1. In FIG. 3a the latter shaped cast (12) symbolizes an organ's vessel structures.

FIG. 2b shows the organ copy that results after completely encasing the negative copy with the open-pore material. The vessel structures (12) are surrounded by foam like material (11).

FIG. 2c schematically shows the end stage, meaning the condition after removal of the negative copy. The final condition of a bioreactor is described in sections and in schematic depictions. FIG. 3 shows the resulting condition when cells (14) are flushed into the open-porous foam structure (11). FIG. 3 shows a spontaneous reorganization in the open-pores of the ceramic foam structure (11) that develops during the perfusion process of culture media through the replicated vessel structures.

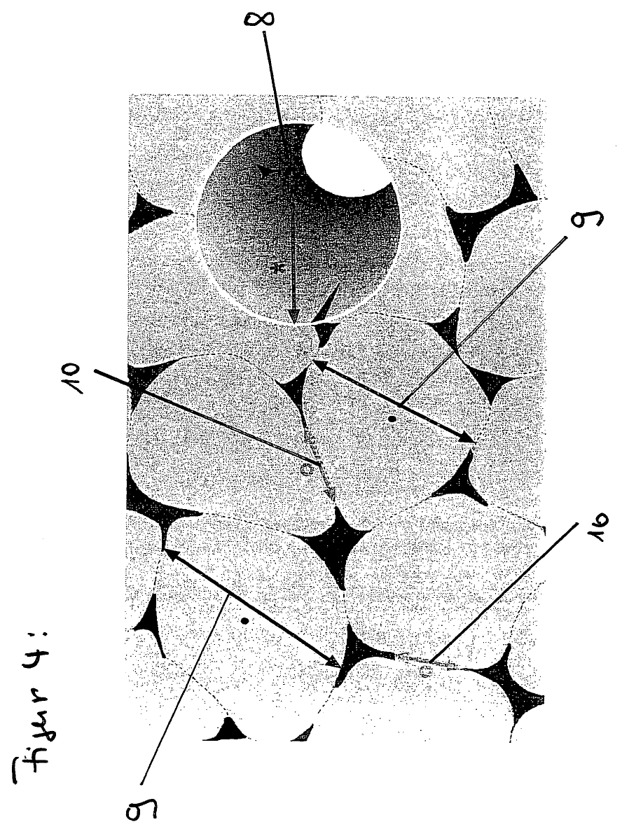

FIG. 4 describes the dimension of the open pore structures: a supply structure (8), that was created by copying a blood vessel; open pores (9) of the foam like material between the copied vascular supply passages; as well as the wall openings between the pores (10) which create the interacting open porous structures.

In FIG. 4 the pore diameter (9) measures 250 micrometer, the open pores measure 100 micrometer, and the supply passage (8) measures the dimension a natural blood capillary. Generally, pore diameters (9) measure 50-1000 micrometer and pore wall openings (10) measure 50-300 micrometer.

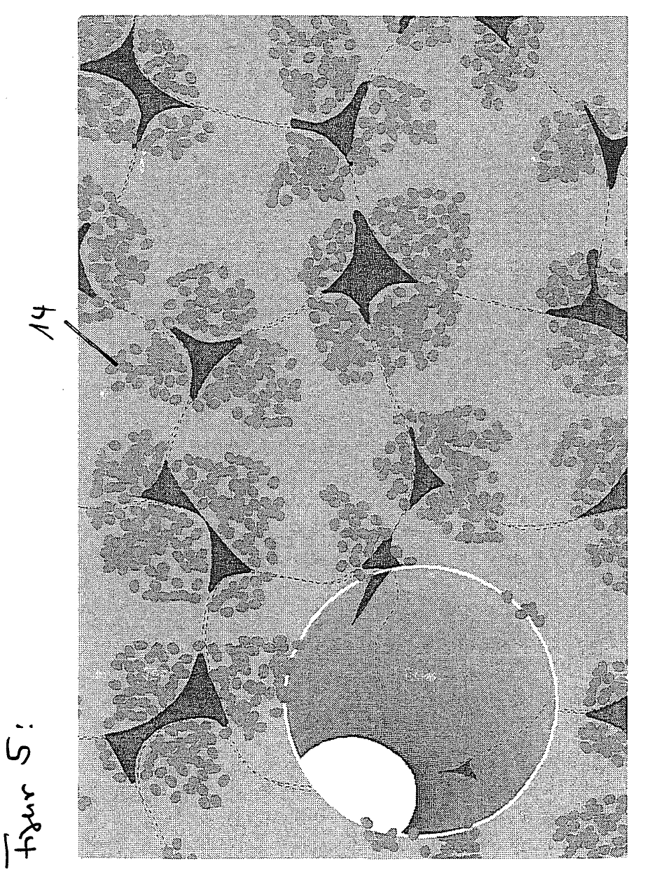

FIG. 5 shows an example referring to FIG. 4 where cells (14) are being seeded.

1. A bioreactor in the form of a perfuseable organ copy. It consists of an immunological, inactive porous body whose open pores consist of organ specific hollow structures that are in communication with each other.

2. Bioreactor according to claim 1:
The distinguishing characteristic of this bioreactor is that the open pores exhibit a diameter of 40-1000 micrometer.

3. Bioreactor according to claim 1 or 2:
The characteristic features are pores that are connected through pore wall openings 10-300 micrometer in diameter.

4. Bioreactor according to at least one of the claims 1-3:
The characteristic feature is that the organ copy is located in a watertight and sterile container and that the container is fitted with connections that are in contact with at least one hollow structure of the organ copy; whereby the container can exhibit various other connections to measure pressure, pH-levels, temperature, or to insert optical probes for microscopies, or to allow measurements inside the module via fluorescent light processes. The container also allows for the sterile opening of the case to remove individual parts of the porous structure that are populated by cells for the purpose of medical implantation.

5. Bioreactor according to at least one of the claims 1-4:
The characteristics are that the container and the connections consist of biodegradable material

6. Bioreactor according to at least one of the claims 1 or 2:
Identifying feature is a porous body that consists of biodegradable material

7. Bioreactor according to at least one of the claims 1 or 2:
Characterized by the fact that the walls of the porous body consist of a sintered ceramic powder.

8. Bioreactor according to claim 3 is identified by the open pore porous body generated from Hydroxyapatite suspension with foam producing additives.

9. Bioreactor according to at least one of the claims 1-5:
Identifying feature is that this bioreactor is a copy of the following organs: bone marrow, lymph nodes, thymus gland, spleen, kidney, pancreas, Islets of Langerhans, mucus membranes, thyroid, parathyroid glands, adrenal glands, bones, testis, uterus, placenta, ovaries, blood vessels, heart, lungs, muscle, heart muscle, intestine, bladder, and/or other mammalian organs.

10. Bioreactor according to at least one of the claims 1-7:
Characterized by the fact that cell lines, immortal cells, primary cells, and/or co-cultures are immobilized inside the open pores.

11. Bioreactor according to claim 8:
Characteristic is that cells are reorganized in high density and/or tissue structure.

12. Process to manufacture a bioreactor in form of an organ copy:
manufacturing of a negative copy of an organ's hollow structures
removal of organic material
surrounding the negative copy with a material that allows the generation of an open pore body
removal of the negative copy
insertion into a sterile and water tight container

13. Process according to claim 12:
Identifying characteristic is that during the generation of the negative copy (manufacturing process a)) a liquid synthetic material is poured into the hollow structure that, after solidification, forms the negative copy.

14. Process according to claim 13:
Characteristic is the use of a two-component-polymer.

15. Process according to claim 13 or 14:
Identifying characteristic is that the chosen synthetic substance disintegrates at a temperature of 600 degrees Celsius.

16. Process according to at least one of the claims 12-15:
Characteristic is that the removal of organic material (process b)) occurs via the use of enzymes like collagenase/trypsin.

17. Process according to at least one of the claims 12-15:
Characteristic is that a chemical treatment with acidic and/or basic substances is applied while removing organic material.

18. Process according to at least one of the claims 12-17:
Characteristic is the creation of an open pore body (process c)) whose pore communicate with each other.

19. Process according to at least one of the claims 12:
Characteristic is that the open pore body has a temperature resistance of maximum 600 degrees Celsius.

20. Process according to at least one of the claims 12-19:
Characteristic is that the open pore body is manufactured from a ceramic powder i.e. Hydorxyapatite and suspensions containing foam producing additives, and that, to some extent, the pore walls brake open during the sintering process.

21. Process according to at least one of the claims 12-20:
Characteristic is that the removal of the negative copy (process d)) occurs via temperature.

22. Process according to at least one of the claims 12-21:
Characteristic is the generation of an open pore body that consists of a material that, after implantation into the body, metabolizes by way of re-absorption into an organ of organic cells.

23. Process according to at least one of the claims 12-22:
Characteristic is the manufacturing of an open pore body that is generated from a biodegradable material that, during the process of in vitro perfusion, forms an organ ex vivo.

24. Process according to at least one of the claims 23:
Characteristic feature is that the generated organ will be transplanted

25. Process according to at least one of the claims 23:
Characteristic is that the container as well as the connections is made from degradable/re-absorbable material that, after transplantation, forms an organ inside the body.

26. Process according to at least one of the claims 12-25:
Characteristic is that animal and/or human organs are used to generate the negative copy of the organ

27. Application of the bioreactor according to one of the claims 1-11:
Breeding, preservation, differentiation, reproduction and/or the use of various and/or individual cell species (co-cultures) of an organ

28. Application according to claim 27 for stem cells, including embryonic stem cells.

29. Application according to claim 27 for human cells

30. Application according to claim 27 for the adult stem of cells

31. Application according to claim 27 for the production of cells

32. Application according to claim 27 for the production of progenitor cells for cell transplantation

33. Application according to claim 27 for the production of gene-technologically modified cells, immortal cells, cell lines, and/or trans-genetic cells

34. Application of the bioreactor according to one of the claims 1-12, for the production of substances through cells

35. Application of the bioreactor according to one of the claims 1-12, for the differentiation of organ typical cells derived from stem cells

36. Application of the bioreactor according to one of the claims 1-12, for the development of organ typical structures from adult stem cells, bone marrow cells or embryonic stem cells.

37. Application of the bioreactor according to one of the claims 1-12 as extracorporeal hybrid organ for organ support

38. Application of the bioreactor according to one of the claims 1-12 as implantable organ transplant

39. Application of the bioreactor according to one of the claims 1-12 as a laboratory and/or supplement for animal research

40. Application of the bioreactor according to one of the claims 1-12 as in vitro virus culture and virus reproduction system

41. Application according to one of the above mentioned claims for the production of the following substances: cellular metabolic products, known or unknown mediators, hormones, differentiation factors, stabilizing factors, signal molecules, growth factors, sensitizing factors, cytokines, proteins, antibodies, vaccines and/or organ specific bio-matrix substances.

42. Application according to one of the above mentioned claims for the development of a hybrid gland.

43. Application according to one of the above mentioned claims for the generation of organic cells like stem cells, differentiated cells of a specific organ, blood cells, and immune cells.

44. Application according to one of the above mentioned claims as hybrid immune system to produce immune competent cells and vaccines, and progenitor cells for organs and blood components.

45. Application according to one of the above mentioned claims as hybrid blood cell system (bone marrow) to produce blood components, especially blood platelets and erythrocytes.

46. Application according to one of the above mentioned claims as hybrid stem cell system to produce progenitor cells for organs, especially to transplant repair cells.

47. Application according to one of the above mentioned claims in cell based therapy, regenerative medicine, cell biology and/or development of vaccines respectively production of vaccines.
